(19) United States
(12) Patent Application Publication
(10) Pub. No.: US 2016/0287700 A1
(43) Pub. Date: Oct. 6, 2016

(54) METHODS, APPARATUSES, AND COMPOSITIONS TO PREVENT INFECTIONS

(71) Applicant: Nasolux LLC, Brooklyn, NY (US)

(72) Inventors: Carter Griffin, Brooklyn, NY (US); Kent Crossley, St. Paul, MN (US)

(21) Appl. No.: 15/084,168

(22) Filed: Mar. 29, 2016

Related U.S. Application Data
(60) Provisional application No. 62/140,040, filed on Mar. 30, 2015.

(51) Int. Cl. A61K 41/00; A61N 5/06; A61K 9/00; A61K 33/18; A61K 31/5415; A61K 33/00

(52) U.S. Cl. CPC A61K 41/0057; A61K 31/5415; A61K 33/00; A61K 9/0043; A61K 33/18; A61N 5/062; A61N 5/0624; A61N 5/0603; A61N 2005/0607; A61N 2005/0662

(57) ABSTRACT

Embodiments of the present technology may include a composition for reducing microorganisms in the anterior nares or at other body sites. The composition may include a photosensitizer and a substance that enhances the eliminating, destroying, killing, or inhibiting of microorganisms in the anterior nares.

### CROSS-REFERENCE TO RELATED APPLICATIONS

This application is a nonprovisional of and claims the benefit of priority to U.S. Provisional Application No. 62/140,040, filed Mar. 30, 2015, the entire contents of which is hereby incorporated by reference for all purposes.

### BACKGROUND

Infections associated with medical care are a major cause of morbidity and mortality. These infections are typically costly to manage and may be associated with a variety of adverse outcomes including death.

Staphylococcus aureus is one of the most common types of bacteria to cause such infections. Most patients who develop infection with this organism are known to be colonized prior to developing clinical infection. Colonization, or asymptomatic carriage of this organism, primarily occurs in the anterior nares (i.e., the anterior part of the nose). Other bacteria that may be acquired as a consequence of medical care, such as a variety of Gram-negative organisms, may also colonize patients and cause clinical infections.

In a medical setting, a patient may transfer S. aureus or other bacteria from a colonization site to a surgical site, wound site, or other parts of the body. Once transferred to these other sites, the organisms may continue to multiply and may develop into a clinical infection.

Photodynamic therapy is the use of light with certain chemicals that are activated by the light energy. This modality may be used in several ways in medicine (e.g., anticancer treatment or antimicrobial therapy). The chemicals that are used (referred to as photosensitizers) may be naturally occurring compounds (e.g., porphyrins, polyynes, psoralens, and anthraquinones), dyes (e.g., methylene blue, Bengal Rose, toluidine blue, rhodamines, etc.), or other unrelated agents (e.g., cyanine compounds) and may have anti-microbial activity alone or on exposure to light or both.

Light energy, which may be between 200 nm and 1400 nm and may include ultraviolet, visible, and infrared light, acts on these chemical substances in different ways to result in an antimicrobial effect. Most often, the effect is to liberate an activated oxygen molecule (so-called singlet oxygen) that is able to destroy both cells and tissues.

For each photosensitizer, there is a corresponding wavelength of light that causes optimal activation. For example, methylene blue may be optimally activated at a wavelength of about 665 nm.

Conventional implementations do not include an additive to a photosensitizer for increasing the effectiveness of the antimicrobial killing in the nares or other sites.

### BRIEF DESCRIPTION OF THE DRAWINGS

FIG. 1 shows a block flow diagram of a method of preventing infections according to embodiments of the present technology.

FIG. 2 shows an apparatus for preventing infections according to embodiments of the present technology.

FIG. 3 shows a nose cone light conductor according to embodiments of the present technology.

### DETAILED DESCRIPTION

Embodiments of the present technology may relate to methods, apparatuses, and compositions for destroying microorganisms in or on the human body. Embodiments may result in inhibiting or eliminating the colonization of the anterior nares with harmful microorganisms such as S. aureus. Embodiments may also improve methods, apparatuses, and compositions to prevent infection where those infections may have originated as a result of colonization of the anterior nares. Embodiments may further enhance the microbial killing effect of photosensitizers.

Embodiments of the present technology may include a composition for reducing microorganisms in the anterior nares or at other body sites. The composition may be a solution, a gel, a cream, a paste, or an emulsion. The composition may include a photosensitizer and a substance that enhances the eliminating, destroying, killing, or inhibiting of microorganisms in the anterior nares. The substance may include an iodide, an iodine, or a nitrite. For example, the iodide may be potassium iodide, sodium iodide, or any iodide comprising a Group I metal. In these and other examples, the iodide may comprise a Group II metal, such as calcium or magnesium. In some embodiments, nitrite may be substituted for iodide. In these and other embodiments, the iodine may be povidone iodine. The composition may include or exclude any compound or group of compounds described. The composition may also exclude solutions that include chlorine, sodium hypochlorite, or hypochlorites.

The substance may enhance the effect of the photosensitizer on the microorganism above an effect otherwise predicted by summing the individual effects of applying the photosensitizer by itself and applying the substance by itself. The photosensitizer, upon activation with light, produces singlet oxygen, which is an energized form of molecular oxygen.

Singlet oxygen is toxic to some microorganisms and may destroy the cell walls or other components of microorganisms. The exact mechanism of the synergistic enhancement of the photosensitizer is not known. The substance may enhance the production of singlet oxygen, may increase the potency of the singlet oxygen, may increase the vulnerability of cell walls to singlet oxygen, may immobilize or mobilize a microorganism, or may act by a combination of these mechanisms or by some other mechanism. Experiments are performed showing that the antimicrobial properties of a mixture of a photosensitizer and a substance are greater than the sum of the antimicrobial properties of the photosensitizer applied separately and any antimicrobial properties of the substance applied separately. For example, using a mixture of the photosensitizer and potassium iodide results in a greater elimination of microorganisms than the sum of the elimination of microorganisms from using the photosensitizer applied by itself and the elimination of microorganisms from using potassium iodide applied by itself.

The photosensitizer may be methylene blue. The wavelength of light that activates the photosensitizer may be between about 600 nm and about 700 nm. The wavelength of light may be a narrower range or may be a single wavelength, such as 665 nm. The photosensitizer may include dimethyl-methylene blue, toluidine blue O, crystal violet, gentian violet, phenothiazinium, phthalocyanine, and perylenequinonoid. The photosensitizer may include or exclude any compound or group of compounds described.

The composition may include singlet oxygen. The singlet oxygen may be present at a concentration higher than under typical ambient conditions. The singlet oxygen may be present at a concentration sufficient to eliminate a significant portion of microorganisms, such as greater than about 50%, greater than about 75%, greater than about 90%, or greater than about 95% of harmful microorganisms in the anterior nares. The composition may include light at a wavelength of between about 600 nm and about 700 nm. The intensity of the light is at an intensity higher than under typical lighting conditions, including under daylight and interior lighting.

The composition may further include a diluent. The diluent may include water or an inert, non-toxic liquid.

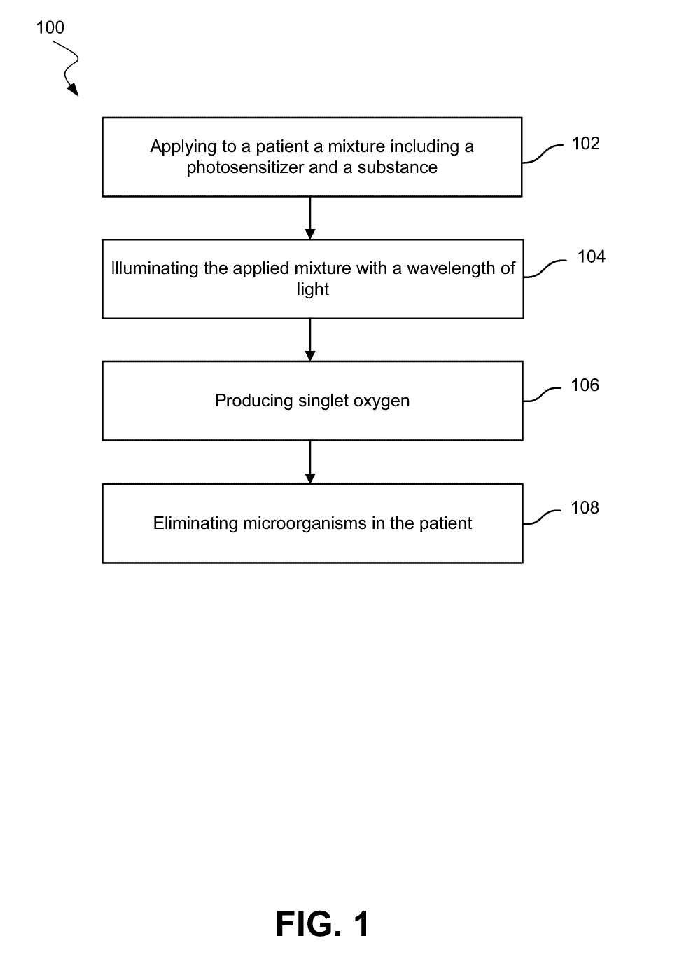

Embodiments of the present technology may include a method of preventing infections. FIG. 1 shows an example method 100. Method 100 may include applying to a patient a mixture of a photosensitizer and a substance 102. The substance may include at least one of an iodide, an iodine, and a nitrite. The substance may be any substance described herein. The mixture may be applied to a nasal passage of the patient. The mixture may be applied to the anterior nares of the patient. The mixture may be applied to both nasal passages of the patient. The mixture may be applied to other sites of colonization or infection. For example, the mixture may be applied to a cutaneous wound.

The mixture may be applied with an applicator. The applicator may be an absorbent swab, an absorbent pad, or an absorbent ball. In some embodiments, the applicator may be a cotton swab, a cotton pad, or a cotton ball. The applicator may include an absorbent material with properties similar to cotton, including blends of cotton and other absorbent materials. The applicator may be a spray, a brush, or a dropper. A combination of applicators may be used to allow the mixture to better reach different regions of the anterior nares. The applicator may be in the nasal passage for between about 5 minutes and about 6 minutes to allow sufficient coverage of the anterior nares. The applicator may also be in the nasal passage for between about 10 seconds to about 20 minutes, about 30 seconds to about 2 minutes, about 1 minute to about 5minutes, about 5 minutes to about 10 minutes, or about 10 minutes to about 20 minutes in embodiments.

Method 100 may include illuminating the applied mixture with a wavelength of light 104. The wavelength of light may be any wavelength of light described herein. Illuminating the applied mixture may include simultaneously illuminating the applied mixture in both nasal passages of the patient. Illuminating the mixture in the patient's nasal passages may be for between about 5 minutes and about 6 minutes. This duration may allow for sufficient activation of the photosensitizer. The duration may depend on the intensity of the light source. The illumination time may also be between about 10 seconds to about 20 minutes, about 30 seconds to about 2 minutes, about 1 minute to about 5 minutes, about 5 minutes to about 10 minutes, or about 10 minutes to about 20 minutes in embodiments.

Method 100 may further include producing singlet oxygen 106. The singlet oxygen may be produced by the illumination of the photosensitizer in the presence of the substance. In addition, method 100 may include eliminating microorganisms in the patient 108. Eliminating microorganisms may include killing, destroying, inhibiting, immobilizing, or rendering harmless the microorganisms. The singlet oxygen may aid in the elimination of the microorganisms.

Embodiments of the present technology may include a nasal applicator. The nasal applicator may include a member having an exterior surface adapted for physiological contact. The member may include a cannula insertable into a nasal passage. The nasal applicator may include a mixture. The mixture may be any mixture described herein. The mixture may be at or in the exterior surface.

The nasal applicator may also include a light conductor extending along the member. The light conductor may have an input at a proximal location along the member and an output at a distal location along the member. The nasal applicator may include a plurality of light conductors, and each light conductor or the plurality of light conductors may have an input at a proximal location along the member and an output at a distal location along the member.

The light conductor may be an optical fiber. The optical fiber may be of any material that can carry light of the desired wavelength. Although substances such as nylon or silica may be used, medical grade plastics (e.g., polytetrafluoroethylene) are preferred because of their relatively low cost. However, if a light source emits ultraviolet light, the fibers should be of glass or quartz because plastics are poor transmitters of ultraviolet light.

The nasal applicator may include a light source. The light source may include a light emitting diode. The nasal applicator may include a light transmitter for transmitting light energy to the input of the light conductor so that the light energy may be conveyed along the member and may be emitted at the output. The light energy may illuminate the exterior surface and the mixture residing on or in the exterior surface. The light energy may be in a wavelength range to activate the photosensitizer so as to release oxygen from the exterior surface. The oxygen may be toxic to microorganisms on or opposite the exterior surface. The oxygen may kill microorganisms or be harmful to microorganisms in any manner previously described.

Embodiments of the present technology, in addition to decolonizing the anterior nares, may kill bacteria and other microorganisms in open wounds that have signs of clinical infection or may be at risk of becoming infected. Embodiments may also be used to treat other types of infection or colonization, including, for example, the ear, pharynx, vagina, etc.

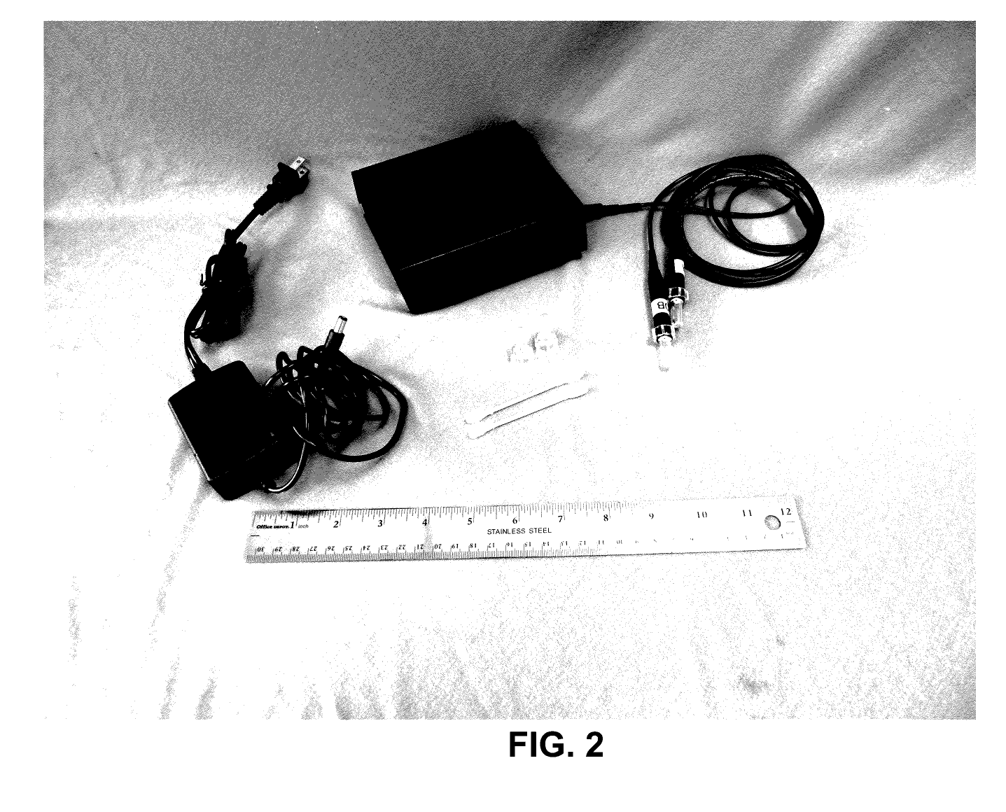

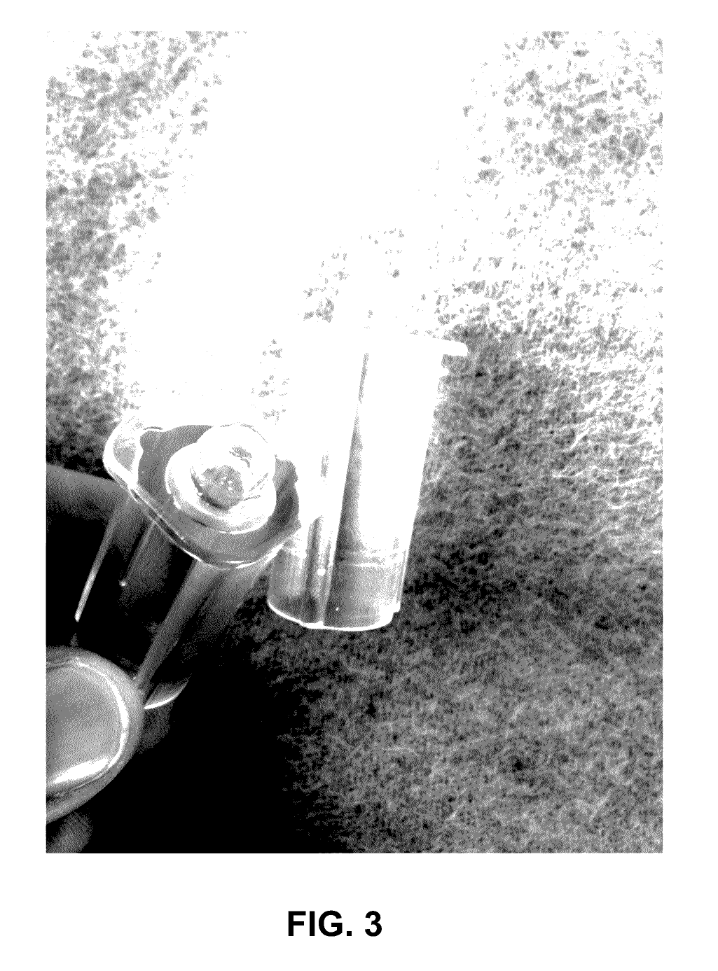

FIG. 2 shows an apparatus for preventing infections according to embodiments of the present technology. Cotton swabs, such as the Q-tip™ swabs shown, may be used to apply a composition to the anterior nares. Light may be illuminated to the anterior nares using light emitting diodes (LEDs) leading to two nose cone light conductors. The power supply for the LEDs may be rechargeable. FIG. 3 shows the nose cone light conductors according to embodiments of the present technology.

In the preceding description, for the purposes of explanation, numerous details have been set forth in order to provide an understanding of various embodiments of the present technology. It will be apparent to one skilled in the art, however, that certain embodiments may be practiced without some of these details, or with additional details.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Additionally, details of any specific embodiment may not always be present in variations of that embodiment or may be added to other embodiments.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a method” includes a plurality of such methods and reference to “the photosensitizer” includes reference to one or more photosensitizer and equivalents thereof known to those skilled in the art, and so forth. The invention has now been described in detail for the purposes of clarity and understanding. However, it will be appreciated that certain changes and modifications may be practice within the scope of the appended claims.

What is claimed is:

1. A composition for reducing microorganisms in the anterior nares or at other body sites, the composition comprising:
a photosensitizer, and
an iodide.

2. The composition of claim 1, wherein the photosensitizer is methylene blue.

3. The composition of claim 1, wherein the iodide comprises potassium iodide.

4. The composition of claim 1, further comprising light of a wavelength between about 600 nm and about 700 nm.

5. The composition of claim 1, further comprising a singlet oxygen.

6. A method of preventing infections, the method comprising:
applying to a patient a mixture comprising a photosensitizer and at least one of an iodide and an iodine;
illuminating the applied mixture with a wavelength of light;
producing singlet oxygen; and
eliminating microorganisms in the patient.

7. The method of claim 6, wherein applying the mixture comprises applying the mixture to the anterior nares of the patient.

8. The method of claim 6, wherein applying the mixture comprises applying the mixture to a wound in the patient.

9. The method of claim 6, wherein applying the mixture comprises the photosensitizer and potassium iodide.

10. The method of claim 6, wherein applying the mixture comprises the photosensitizer and povidone iodine.

11. The method of claim 6, wherein the photosensitizer comprises methylene blue.

12. The method of claim 6, wherein the wavelength of light is between about 600 nm and about 700 nm.

13. The method of claim 6, wherein illuminating the applied mixture comprises simultaneously illuminating the applied mixture in two nasal passages of the patient.

14. The method of claim 11, wherein illuminating the applied mixture comprises illuminating the applied mixture for between about 5 minutes and about 6 minutes.

15. The method of claim 11, wherein illuminating the applied mixture comprises illuminating the applied mixture for between about 10 second and about 20 minutes.

16. A nasal applicator, the nasal applicator comprising:
a member having an exterior surface adapted for physiological contact and comprising a cannula insertable into a nasal passage;
a mixture comprising a photosensitizer and at least one of an iodide and an iodine at or in the exterior surface;
a light conductor extending along the member, the light conductor having an input at a proximal location along the member and an output at a distal location along the member so that light energy at the input is conveyed along the member and is emitted at the output so as to illuminate the exterior surface and the mixture residing on or in the exterior surface, the light energy being in a wavelength range to both activate the photosensitizer so as to release oxygen from the exterior surface, wherein the oxygen is toxic to microorganisms on or opposite the exterior surface.

17. The nasal applicator of claim 16, wherein the photosensitizer is methylene blue.

18. The nasal applicator of claim 16, wherein the mixture comprises a photosensitizer and potassium iodide.

19. The nasal applicator of claim 16, wherein the oxygen kills microorganisms.
